(19) United States
(12) Patent Application Publication
(10) Pub. No.: US 2026/0346049 A1
(43) Pub. Date: Nov. 5, 2026

(54) SYSTEMS AND METHODS TO DETERMINE PRESCRIPTION DOSE BASED ON OUTCOME

(71) Applicants: MIM SOFTWARE INC., CLEVELAND, OH (US); CVERGENX, INC., ST. PETERSBURG, FL (US)

(72) Inventors: JACOB SCOTT, GATES MILLS, OH (US); JAVIER TORRES-ROCA, ST. PETERSBURG, FL (US); JONATHAN PIPER, ORANGE, OH (US); ELLIOTT ABBOTT, BOSTON, MA (US); DANE PITTOCK, WILLOUGHBY, OH (US)

(21) Appl. No.: 19/665,768

(22) Filed: May 1, 2026

Related U.S. Application Data
(60) Provisional application No. 63/799,133, filed on May 2, 2025.

(51) Int. Cl. A61N 5/10; G16H 20/40

(52) U.S. Cl. CPC A61N 5/1031; G16H 20/40; A61N 2005/1032

(57) ABSTRACT

Systems and associated methods are provided for selecting a prescription dose based on outcome. Cancer outcomes of a population of patients can be determined. Based on the outcomes for the population, a tumor control metric related to dose can be derived. The tumor control metric can be related to genomics and/or tumor biology. A treatment plan optimization can be performed to optimize a prescription dose to determine a treatment plan.

### CROSS-REFERENCE TO RELATED APPLICATIONS

This application claims priority to U.S. Provisional Patent Application Ser. No. 63/799,133, filed on May 2, 2025. The entirety of the aforementioned application is incorporated herein by reference.

### BACKGROUND

Radiation therapy is a common treatment in oncology. Radiation therapy delivery is physically accurate, but often biologically imprecise. That is, a physical dose is readily measured, but biological effect is more challenging to understand. To date, personalized radiation therapy has meant anatomic personalization (i.e. dose shape).

### SUMMARY

A simplified summary is provided herein to help enable a basic or general understanding of various aspects of exemplary, non-limiting embodiments that follow in the more detailed description, the accompanying drawings, and the attached appendices. This summary is not intended, however, as an extensive or exhaustive overview. Instead, the sole purpose of the summary is to present some concepts related to some exemplary non-limiting embodiments in a simplified form as a prelude to the more detailed description of the various embodiments that follow.

In various, non-limiting embodiments, a system and associated methods are provided for selecting a prescription dose based on outcome. Cancer outcomes of a population of patients can be determined. Based on the outcomes for the population, a tumor control metric related to dose can be derived. The tumor control metric can be related to genomics and/or tumor biology. A treatment plan optimization can be performed to optimize a prescription dose to determine a treatment plan.

In one implementation, a method is provided. The method includes establishing a population genomic adjusted radiation dose (GARD) model. The method further includes determining a metric indicative of an improvement in outcome relative to dose. In addition, the method include generating a treatment plan that includes at least a prescription dose based on an optimization with respect to outcome.

In another implementation, a system is provided. The system includes a processor coupled to memory storing computer-executable instructions. The instructions, when executed by the processor, configure the processor to: establish a population-based model of genomic adjusted radiation dose (GARD); determine a metric, based on the population-based model, indicative of one of an improvement or deterioration in outcome relative to a change in dose; and generate a treatment plan that includes at least a prescription dose based on an optimization based on the metric and with respect to outcome.

In yet another implementation, a non-transitory, computer-readable storage medium is provided. The computer-readable storage medium stores computer-executable instructions. The instructions, when executed by a processor, configure the processor to: establish a population-based model of genomic adjusted radiation dose (GARD) based a distribution of respective GARD values for patients of a population; determine a metric, based on the population-based model, indicative of one of an improvement or deterioration in outcome relative to a change in dose; generate a treatment plan that includes at least a prescription dose based on an optimization based on the metric and with respect to outcome; and output the prescription dose to a radiotherapy system.

To the accomplishment of the foregoing and related ends, the following description and annexed drawings set forth certain illustrative aspects and implementations. These are indicative of but a few of the various ways in which one or more aspects may be employed. Other aspects, advantages and features of the disclosure will become apparent from the following detailed description when considered in conjunction with the annexed drawings.

### BRIEF DESCRIPTION OF THE DRAWING

Various non-limiting embodiments are further described in the detailed description given below with reference to the accompanying drawings, which are incorporated in and constitute a part of the specification.

FIG. 1 is a flow diagram of an exemplary, non-limiting method for selecting a prescription dose based on a likelihood of outcome in accordance with various aspects.

FIG. 2 is a flow diagram of an exemplary, non-limiting method for determining a population-based GARD model according with one or more aspects.

FIG. 3 is an exemplary, non-limiting GARD distribution for a population according to various aspects.

FIG. 4 is an exemplary, non-limiting treatment plan report for head and neck cancer in accordance with various aspects.

FIG. 5 is an exemplary, non-limiting treatment plan report for breast cancer in accordance with one or more aspects.

FIG. 6 is a block diagram of an exemplary, non-limiting system for selecting a prescription dose based on a likelihood of outcome according to one or more aspects.

FIG. 7 is a schematic block diagram of an exemplary, non-limiting embodiment for a computing device with which aspects described herein may be implemented.

### DETAILED DESCRIPTION

As described above, radiation therapy is a common therapeutic agent in oncology. The practice is often empiric. Further, while conventional radiation therapy delivery is physically accurate, it is biologically imprecise. Radiation therapy is often planned to configure, anatomically, dose shape. For instance, three-dimensional conformal radiation therapy (3D-CRT) is a process to plan radiation therapy, such as external beam ration therapy (EBRT). A 3D view of a tumor (and sometimes nearby healthy tissue) is acquired. Using the view, a size and shape of a tumor can be determined and radiation is configured to conform to a fit the tumor. Intensity-modulated radiation therapy (IMRT) is another technique that shapes beams of radiation to a shape of a tumor.

Genomic-driven oncology is a growing trend with promising results. Tumor heterogeneity can be measured by more than just mutations. For example, other factors include gene expression, protein expression, copy number variation, epigenetics, microenvironment, or the like. Studies have shown that an estimated 25% of cancer patients in the United States could benefit from genome-driven care. One technique for breast cancer tests a sample to look for a group of genes indicative of a benefit from chemotherapy. That is, the technique can identify a first percentage of patients who may not receive a benefit from chemotherapy and, accordingly, a second percentage of patients that will. Another technique utilizes a 70-gene signature result to a proportion of patients for which systemic chemotherapy is recommended.

With radiation therapy, dose is measured but conventional techniques do not factor the biologically effect of the dose on a patient. For example, an identical dose delivered to different patients can result in greatly varying biological effects. Through combination of a gene expression (e.g. molecular scale) and cellular radiosensitivity (e.g. cellular scale) through a mathematical expression (e.g. pathway scale) can lead to biological effect at the organism scale. Such efforts attempt to build a radiation sensitivity network to facilitate biomarker discovery. Linear regression on such a radiation sensitivity network yields a radiation sensitivity index (RSI). In some examples, RSI derives from expression levels of one or more genes including androgen receptor (AR), jun oncogene (c-Jun), signal transducer and activator of transcription 1 (STAT1), protein kinase C, beta (PKCb), V-rel reticuloendotheliosis viral oncogene homolog A (RELA or p65), c-Abl oncogene 1 (c-Abl); small ubiquitin-like modifier 1 (SUMO1), p21 activated kinase-2 (PAK2), histone deacetylase 1 (HDAC1), and interferon regulatory factor 1 (IRF1). RSI has shown to operate as an indicator of acceptable radiation response. That is, RSI alone can operate similar to the metrics describes above for chemotherapy.

In a further aspect, RSI is a continuous value. That is, RSI is heterogeneous across and between disease sites, is bimodal, and can span most of a range between 0 and 1. Leveraging this, a personalized radiation dose can be derived based on a linear quadratic equation of radiation effect generally given by the following:

SF=e^{-n(αd+βd}^{²⁾},

where n is a number of fractions of radiation, d is the dose per fraction, and α and β are linear and quadratic radiosensitivity parameters, respectively. RSI, according to an aspect, represents a survival fraction at 2 Gy in cell lines. Accordingly, by substituting RSI for SF above, a patient-specific α can be determined according to the following:

$$α = \frac{ln RSI + β {nd}^{2}}{- nd} .$$

A genomic adjusted radiation dose (GARD) can be derived from an equation for biologic effect, the patient-specific α, and the radiation dose and fractionation schedule received by the patient. GARD demonstrates that a fixed physical dose does not always provide equivalent effect. GARD is associated with survival and recurrence. For example, for a unit increase in GARD, a decrease results in relative hazard and overall survival.

As shown above, GARD quantifies a personalized effect of a given radiation prescription. This effect is a statistically significant predictor of outcome and is actionable by dose modulation. In addition to personalized prediction, GARD provides predicative functions at a population level. A predetermined or target GARD value can be utilized to partition a survival curve into two groups. Specifically, a value, which can also represent a personalized radiation dosage (RxRSI), is derived from the target GARD value and can operate to identify patients receiving a biologically adequate dose or above and patients an underdose biologically. In other words, over/under dosing can be quantified in terms of biological effect.

As described above, conventional treatment planning systems often seek to satisfy normal tissue dose constraints to achieve a specified and fixed physical prescription dose (Gy). A prescription dose is conventionally selected based on gestalt and/or within standard of care dose ranges.

Further, as discussed above, genomic-driven oncology is advancing. In radiation therapy, one example of personalized, genomic-driven treatment can involve determining a radiation sensitivity index (RSI) of a tumor based on, for example, expression levels of one or more signature genes in the tumor. A personalized dose can be calculated based on a predetermined genomic adjusted radiation dose (GARD) and the RSI. In some examples, the GARD can be determined based the RSI and a radiation dose and fractionation schedule.

Systems and methods are provided herein to select a prescription dose based on a likelihood to improve outcomes. In some examples, the likelihood to improve outcomes is weighed against normal tissue constraints. Normal tissue constraints, in an aspect, are indicative of a potential for normal tissue complications due to dose. In an exemplary implementation, cancer outcomes (e.g., progression free survival) and radiosensitivity (e.g. GARD) of a population of patients are determined. Based on these determinations, a tumor control metric related to dose is derived. For instance, in one implementation, the population can be segregated into different modes such as, for example, two sub-populations. The sub-populations can include one set including patients receiving highly effective doses given respective personal radiosensitivities (GARD-HI) and another set including patients who receiving relatively less effective doses (GARD-LO). A proportion of patients (e.g. % GARD-HI) falling into the highly effective group (e.g. GARD-HI) can be calculated at each physical prescription dose, which in some examples is given in grays (Gy). Using these data, a treatment plan optimization engine, such as a multi-criteria optimizer (MCO), can optimize prescription dose. The prescription dose can be optimized, in some implementations, along with other constraints or weighted metrics such as but not limited to normal tissue dose constraints or normal tissue complication probability (NTCP). The treatment plan optimization engine can generate an optimal treatment plan or a set of optimal plans (e.g., the pareto optimal plans in an MCO). In other examples, other surrogates for a metric relate to a dose may be utilized. For example, a tumor control probability (TCP) can be determined.

The claimed subject matter is now described with reference to the drawings, where like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details.

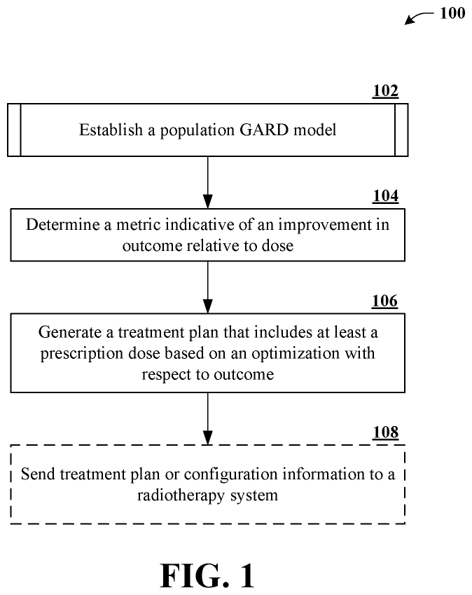

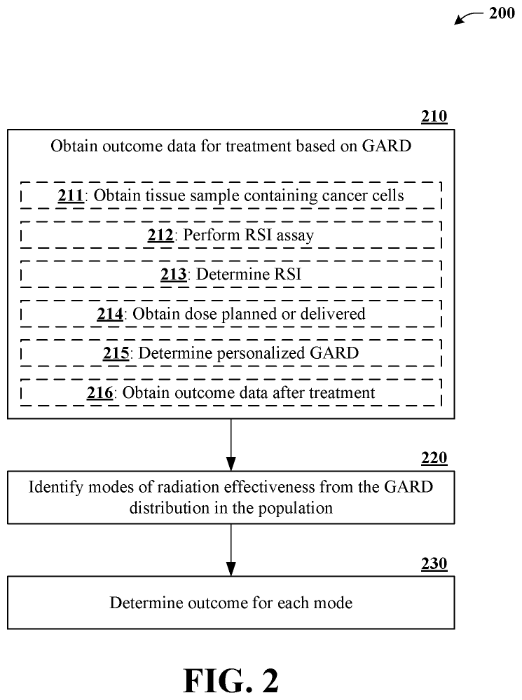

Referring initially to FIG. 1, illustrated is a flow diagram of an exemplary, non-limiting method 100 for selecting a prescription dose based on a likelihood of outcome. Method 100 can begin at 102, where a population-based GARD model is established. For example, method 200, shown in FIG. 2, provides one exemplary implementation for determining the population-based GARD model. At 210, outcome data for treatment based on GARD can be obtained. In one exemplary embodiment, obtaining outcome data for treatment based on GARD can include obtaining outcome data for each patient in a population of patients. For instance, for a given patient, the process can include obtaining a tissue sample containing cancer cells, as shown at 211. At 212, an genomic assay is performed. At 213, based on the assay, RSI is determined. At 214, a dose planned for or delivered to the patient is obtained. At 215, a personalized GARD for the patient is determined based on the RSI and the dose. At 216, outcome data after treatment is obtained. For example, outcome data can include a time to recurrence, progression, death, etc.

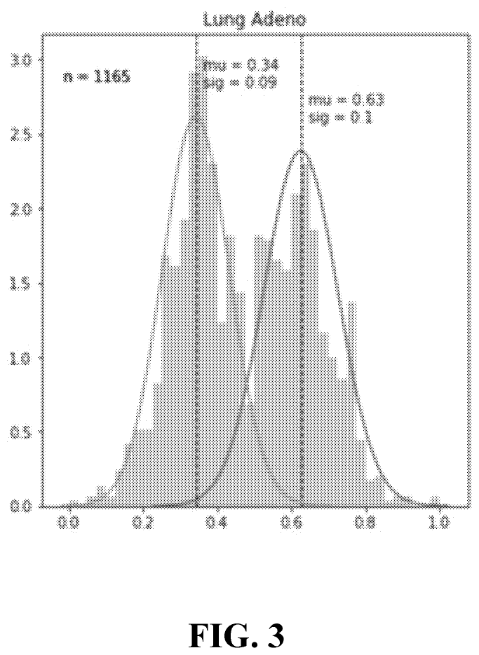

With outcome data for each patient obtained, modes of radiation effectiveness can be identified, at 220, from a GARD distribution in the population. In one example, the modes can be identified by splitting the population according to median GARD. This provides a first group of patients who received a GARD higher than the median and a second group of patients who received a GARD lower than the median. In another example, the modes can be identified by decomposing the distribution to determine a cut-off GARD. The cut-off GARD may be a value maximizing separation between groups as shown in FIG. 3. In this example, a first group of patients is identified as patients receiving a dose (or effect) yielding GARD higher than the cut-off. A second group of patients is identified as patients receiving a dose yielding GARD (or effect) lower than the cut-off. It is to be appreciated that other modes can be identified in the population. Further, the model can be a continuous model rather than a cut-off based model.

At 230, the outcomes associated with each mode identified are characterized. For example, the outcome can be characterized by a Kaplan-Meier curve. In another example, the outcome can be characterized by a percentage of patients whose disease progresses earlier than 5 years (or other time point) post treatment.

Referring again to FIG. 1, at 104, a metric indicative of an improvement in outcome, relative to dose, is determined. In some implementations, the metric can be, but is not limited to, a differentiable metric enabling gradient optimizers to be used scalably. The metric, in some examples, can be a surrogate for an improvement in outcomes related to an increase in physical dose (Gy). The outcomes can be biologically-derived or GARD-derived outcomes, Accordingly, the outcomes can be a GARD-derived outcome predictions. A dose, in Gy, within a dose range of the population evaluated in step 102 has a derivable GARD-metric. For instance, in a non-limiting, exemplary embodiment, for a given radiation therapy dose input, the metric can be a percentage is determined where the percentage indicates a proportion of patients in the population receiving a dose yielding a GARD greater than or equal to a threshold value. In some implementations, the threshold value can be the cut-off described above.

In a further example, expectation maximization can be utilized, which models a population distribution as a mixture of Gaussian distributions to determine respective means and variances. The threshold value, for example, can be determined based on the means and variances. For instance, the threshold value can be determined based on where a probability is equal that a particular sample belongs to either distribution.

At 106, a treatment plan is generated. In an exemplary implementation, the treatment plan includes at least a prescription dose based on an optimization with respect to outcome. According to one example, a multi-criteria optimization is performed to identify pareto-optimal surfaces, which allows a clinician to select an optimal trade-off between normal tissue doses (or effects) and tumor dose (or effect). In another example, a weighting between normal tissue complication probability (NTCP) and tumor control probability (TCP) (or a value expressed in the metric above) is defined. An optimization engine determines an optimal treatment plan, given the weighting. In yet another example, a weighting between normal tissue dose constraints (such as, but not limited to, a percentage of an organ receiving more than a given dose) and tumor control probability is defined. In some implementations, tumor control probability is expressed based on the metric above). An optimization engine determines an optimal treatment plan, given the weighting and subject to satisfaction of the constraints.

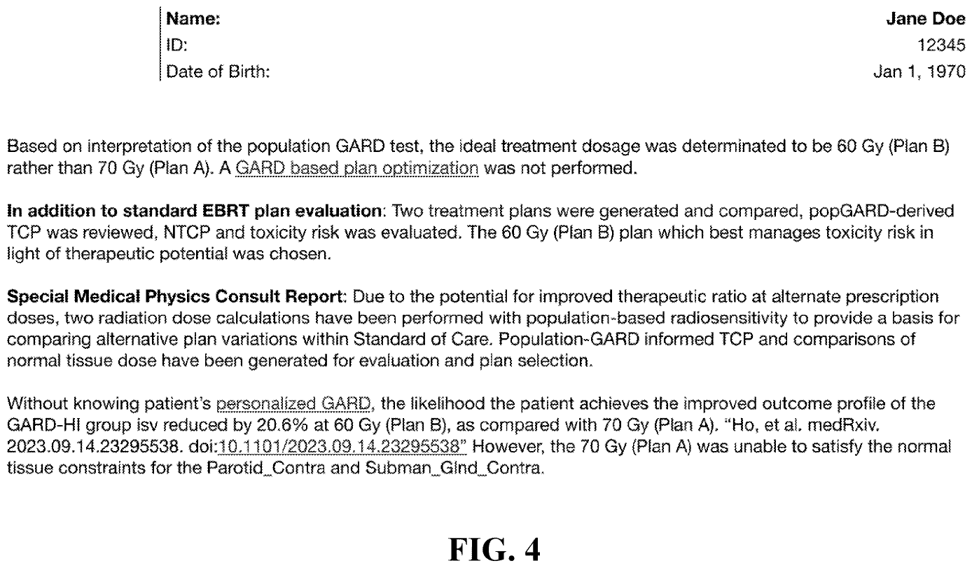

In accordance with one example, a treatment plan report for head and neck cancer, according to one or more aspects, is shown in FIG. 4.

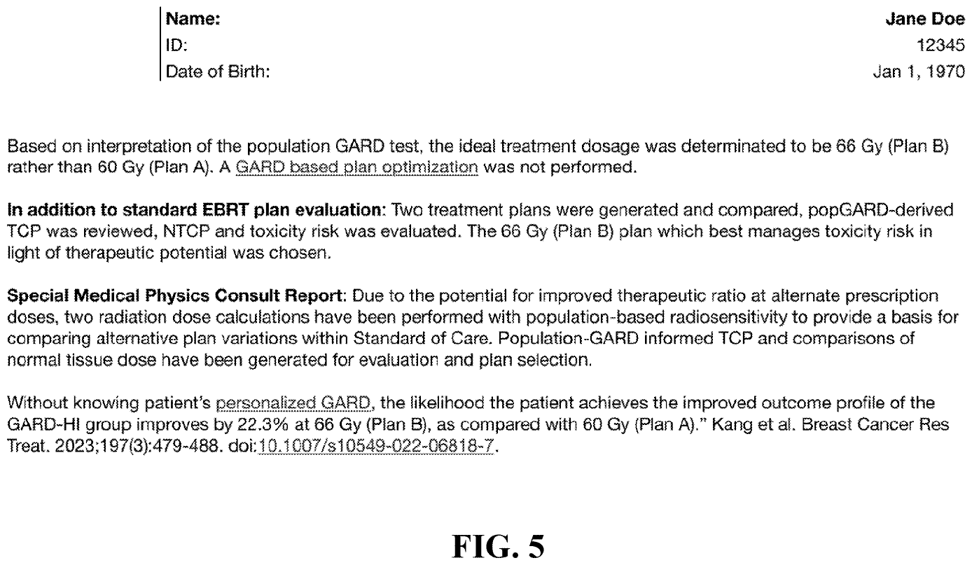

In accordance with another example, a treatment plan report for breast cancer, according to one or more aspects, is shown in FIG. 5.

In a further aspect, at 108, the generated treatment plan or configuration information based on the generated treatment plan can be output or exported for a radiotherapy system. Accordingly, the radiotherapy system can be configured to emit treatment radiation according to target zones and prescription dose indicated in the generated treatment plan.

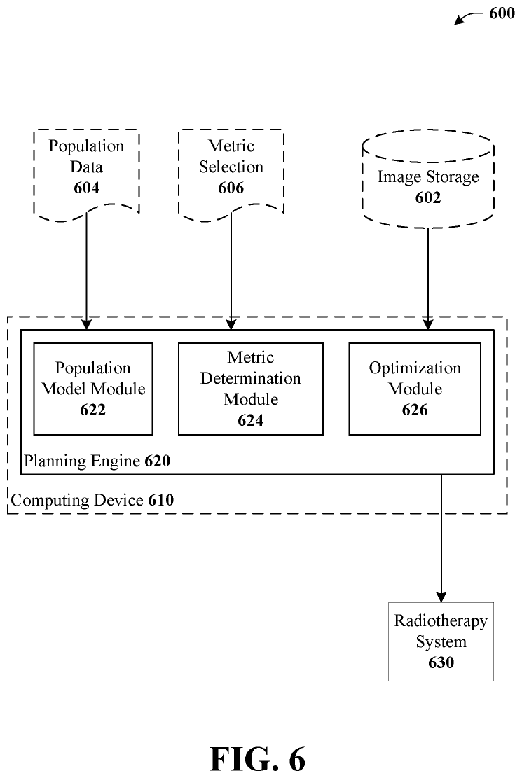

Turning to FIG. 6, illustrated is a block diagram an exemplary, non-limiting system 600. System 600, in an aspect, can implement the methods depicted in FIGS. 1-2 and described above. System 600 includes a computing device 610, which can have a processor and a memory, configured to implement a planning engine 620. Planning engine 620 can include various functional modules implemented by computer-executable instructions. The modules can include a population model module 622, a metric determination module 624, and an optimization module 626.

As shown in FIG. 6, population data 602 can be obtained. The population model module 622 utilizes the population data 602 to generate a population GARD model, for example. The population data 602, in an aspect, can include RSI for patients of a population, planned or received dose for patients, outcome data for patients of the population, etc. For example, for each patient in a population, outcome data for treatment based on GARD can be obtained. Various modes of radiation effectiveness from the GARD distribution of the population can be identified. For each mode, identified, respective outcomes can be characterized to provide the model.

Metric determination module 624 can determine a metric based on the population GARD model. In some implementation, input, such as a metric selection, can facilitate metric determination. The metric can be indicative of an improvement in outcome, relative to dose. The metric, in some examples, is a surrogate for an improvement in outcomes related to an increase in physical dose (Gy). For instance, for a given radiation therapy dose input, the metric determined can be a percentage is determined where the percentage indicates a proportion of patients in the population receiving a dose corresponding to a GARD greater than or equal to a threshold value.

Optimization module 626 can determine at least a prescription dose based on an optimization with respect to outcome. As some optimization techniques utilize normal tissue effects, a normal tissue complication probability, or other normal tissue constraints, image data from an image system 602 can be acquired. The image data, in an aspect, can include one or more contours of a tumor and/or normal tissue. The image data can facilitate determination of constraints relative to a specific case.

According to one example, the optimization module 626 can perform a multi-criteria optimization to identify pareto-optimal surfaces, which allows a clinician to select an optimal trade-off between normal tissue doses (or effects) and tumor dose (or effect).

In another example, optimization module 626 and/or metric determination module 624 defines a weighting between normal tissue complication probability (NTCP) and tumor control probability (TCP) (or a value expressed in the metric above). The optimization module 626 determines an optimal treatment plan, given the weighting.

In yet another example, optimization module 626 and/or metric determination module 624 defines a weighting between normal tissue dose constraints and tumor control probability. The optimization module 626 determines an optimal treatment plan, given the weighting and subject to satisfaction of the constraints.

In a further aspect, planning engine 620 can output or send the prescription dose, or configuration information bases thereon, to a radiotherapy system 630. The radiotherapy system 630 can be configured to emit treatment radiation according to target zones from image data and/or prescription dose.

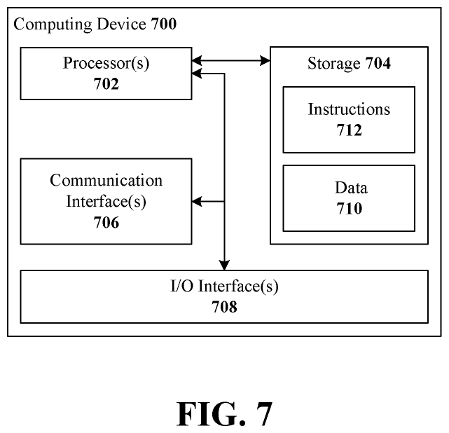

FIG. 7 illustrates a schematic block diagram of an exemplary, non-limiting embodiment for a computing device 700 with which aspects of disclosed herein can be implemented. As shown in FIG. 7, computing device 700 includes one or more processor(s) 702 configured to execute computer-executable instructions 712 such as instructions for carrying out one or more aspects described herein. Such computer-executable instructions 712 can be stored on one or more computer-readable media including non-transitory, computer-readable storage media such as storage 704. For instance, storage 704 can include non-volatile storage to persistently store instructions 712 and/or data 710 (e.g., models, population data, outcomes, weights, metrics, working data, etc.). Storage 704 can also include volatile storage that stores instructions 712 and other data 710 (or portions thereof) during execution by processor 702.

Computing device 700 includes a communication interface 706 to couple computing device 700 to various remote systems (e.g. treatment systems, etc.). Communication interface 706 can be a wired or wireless interface including, but not limited, a WiFi interface, an Ethernet interface, a fiber optic interface, a cellular radio interface, a satellite interface, etc. An I/O interface 708 is also provided to couple computing device 700 to various input and output devices such as displays, touch screens, keyboards, mice, touchpads, etc. By way of example, I/O interface 708 can include wired or wireless interfaces such as, but not limited to, a USB interface, a serial interface, a WiFi interface, a short-range RF interface (Bluetooth), an infrared interface, a near-field communication (NFC) interface, etc.

According to an aspect, a method is provided. The method includes establishing a population genomic adjusted radiation dose (GARD) model. The method further includes determining a metric indicative of an improvement in outcome relative to dose. In addition, the method includes generating a treatment plan that includes at least a prescription dose based on an optimization with respect to outcome.

In an example, the GARD model is based on a radiation sensitivity index (RSI) and a radiation dose and fractionation schedule. In another example, the metric includes a percentage, where the percentage indicates a proportion of patients in the population receiving a dose corresponding to a GARD greater than or equal to a threshold value.

In another example, the threshold value is a median GARD value in a GARD distribution for the population. In another example, establishing the population GARD model includes obtaining at least one tissue sample containing cancer cells, performing radiosensitivity index genomic assay, computing the radiosensitivity index, administering the radiation therapy dose to the cancer cells, and obtaining outcome data after treatment.

In another example, establishing the population GARD model includes identifying modes of radiation effectiveness from a GARD distribution in the population. Further to this example, the metric is derived from a GARD value determined based on the modes of radiation effectiveness identified from the GARD distribution. In an example, the method further includes determining the GARD value so that a separation between at least two modes is maximized. In another example, establishing the population GARD model includes characterizing the outcomes of each mode.

In an example, the method further includes using an optimizer to optimize a treatment plan and prescription dose based on the metric. In an example, the optimizer performs a multi-criteria optimization to enable balance between normal tissue effects and tumor effects. In another example, the optimizer defines a weighting between normal tissue complication probability and tumor control probability. In another example, the optimizer defines a weighting between normal tissue dose constraints and tumor control probability, wherein the optimization is based on the weighting subject to satisfaction of the constraints.

In an example, the method further includes exporting the prescription dose to a radiotherapy system.

In another aspect, a system is provided. The system includes a processor coupled to memory storing computer-executable instructions. The instructions, when executed by the processor, configure the processor to: establish a population-based model of genomic adjusted radiation dose (GARD); determine a metric, based on the population-based model, indicative of one of an improvement or deterioration in outcome relative to a change in dose; and generate a treatment plan that includes at least a prescription dose based on an optimization based on the metric and with respect to outcome.

In an example, the population-based model is based on population data generated by, for each patient in a population, obtaining at least one tissue sample containing cancer cells, performing radiosensitivity index genomic assay, computing the radiosensitivity index, obtaining a radiation therapy dose to the cancer cells, and obtaining outcome data after treatment.

In another example, the metric includes a percentage, where the percentage indicates a proportion of patients in the population receiving a dose corresponding to a GARD greater than or equal to a threshold value.

In another example, the processor is further configured to identify modes of radiation effectiveness from a GARD distribution and determine the threshold value based on the modes identified.

In another example, the processor is further configured to export the prescription dose to a radiotherapy system.

In yet another aspect, a non-transitory, computer-readable storage medium is provided. The medium stores computer-executable instructions. The instructions, when executed by a processor, configure the processor to: establish a population-based model of genomic adjusted radiation dose (GARD) based a distribution of respective GARD values for patients of a population; determine a metric, based on the population-based model, indicative of one of an improvement or deterioration in outcome relative to a change in dose; generate a treatment plan that includes at least a prescription dose based on an optimization based on the metric and with respect to outcome; and output the prescription dose to a radiotherapy system.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such features may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

1. A method comprising:
establishing a population genomic adjusted radiation dose (GARD) model;
determining a metric indicative of an improvement in outcome relative to dose; and
generating a treatment plan that includes at least a prescription dose based on an optimization with respect to outcome.

2. The method of claim 1, wherein the GARD model is based on a radiation sensitivity index (RSI) and a radiation dose and fractionation schedule.

3. The method of claim 1, wherein the metric includes a percentage, where the percentage indicates a proportion of patients in the population receiving a dose corresponding to a GARD greater than or equal to a threshold value.

4. The method of claim 3, wherein the threshold value is a median GARD value in a GARD distribution for the population.

5. The method of claim 1, wherein establishing the population GARD model includes obtaining at least one tissue sample containing cancer cells, performing radiosensitivity index genomic assay, computing the radiosensitivity index, administering the radiation therapy dose to the cancer cells, and obtaining outcome data after treatment.

6. The method of claim 1, wherein establishing the population GARD model includes identifying modes of radiation effectiveness from a GARD distribution in the population.

7. The method of claim 6, wherein the metric is derived from a GARD value determined based on the modes of radiation effectiveness identified from the GARD distribution.

8. The method of claim 6, further comprising determining the GARD value so that a separation between at least two modes is maximized.

9. The method of claim 6, wherein establishing the population GARD model includes characterizing the outcomes of each mode.

10. The method of claim 1, further comprising using an optimizer to optimize a treatment plan and prescription dose based on the metric.

11. The method of claim 10, wherein the optimizer performs a multi-criteria optimization to enable balance between normal tissue effects and tumor effects.

12. The method of claim 10, wherein the optimizer defines a weighting between normal tissue complication probability and tumor control probability.

13. The method of claim 10, wherein the optimizer defines a weighting between normal tissue dose constraints and tumor control probability, wherein the optimization is based on the weighting subject to satisfaction of the constraints.

14. The method of claim 1, further comprising exporting the prescription dose to a radiotherapy system.

15. A system, comprising:
a processor coupled to memory storing computer-executable instructions that, when executed by the processor, configure the processor to:
establish a population-based model of genomic adjusted radiation dose (GARD);
determine a metric, based on the population-based model, indicative of one of an improvement or deterioration in outcome relative to a change in dose; and
generate a treatment plan that includes at least a prescription dose based on an optimization based on the metric and with respect to outcome.

16. The system of claim 15, wherein the population-based model is based on population data generated by, for each patient in a population, obtaining at least one tissue sample containing cancer cells, performing radiosensitivity index genomic assay, computing the radiosensitivity index, obtaining a radiation therapy dose to the cancer cells, and obtaining outcome data after treatment.

17. The system of claim 15, wherein the metric includes a percentage, where the percentage indicates a proportion of patients in the population receiving a dose corresponding to a GARD greater than or equal to a threshold value.

18. The system of claim 17, wherein the processor is further configured to:
identify modes of radiation effectiveness from a GARD distribution; and
determine the threshold value based on the modes identified.

19. The system of claim 15, wherein the processor is further configured to export the prescription dose to a radiotherapy system.

20. A non-transitory, computer-readable storage medium having stored thereon computer-executable instructions that, when executed by a processor, configure the processor to:
establish a population-based model of genomic adjusted radiation dose (GARD) based a distribution of respective GARD values for patients of a population;
determine a metric, based on the population-based model, indicative of one of an improvement or deterioration in outcome relative to a change in dose;
generate a treatment plan that includes at least a prescription dose based on an optimization based on the metric and with respect to outcome; and
output the prescription dose to a radiotherapy system.
